(19) United States
(12) Patent Application Publication
(10) Pub. No.: US 2026/0182984 A1
(43) Pub. Date: Jul. 2, 2026

(54) SUTURING DEVICE

(71) Applicant: Durastat LLC, Austin, TX (US)

(72) Inventors: Kevin T. Foley, Memphis, TN (US); Adam Azzara, Austin, TX (US); Jens Johnson, Austin, TX (US); Michael J. Milella, JR., San Diego, CA (US); Jeremy Markovich, San Diego, CA (US)

(21) Appl. No.: 19/002,881

(22) Filed: Dec. 27, 2024

(51) Int. Cl. A61B 17/06; A61B 17/00; A61B 17/04

(52) U.S. Cl. CPC A61B 17/06061; A61B 17/0482; A61B 2017/00367; A61B 2017/06052

(57) ABSTRACT

A suturing device includes a cannula, a needle pusher and an actuator mechanism. The cannula includes a needle compartment defining a needle-receiving passage terminating at a distal opening of the cannula and a notch extending from the distal opening into the cannula. The needle pusher includes a wire provided in the cannula. The actuator mechanism operably connects with the needle pusher such that actuation of the actuator mechanism results in movement of the wire within the cannula toward the distal opening.

### BACKGROUND

The present disclosure relates generally to surgery and the placement of sutures, and more particularly to devices and methods for suture repair of the tissue.

Surgical closure using sutures is one approach to tissue repair. Oftentimes a needle driver or similar device is used to locate and to pass a suture needle through the tissue to be repaired. The suture needle attaches at one end to a predetermined length of suture, which can be stored in a suture package. Other wound closure devices, such as staples, and other repair devices, like mesh or patch reinforcements, are frequently used for repair.

Locating the tissue repair devices, especially when working in areas where it is difficult for a surgeon to access such as when working through a tubular retractor or other portal, can be very challenging. U.S. Pat. No. 10,610,215 B2 discloses a suturing device that includes an elongate body, a needle holder, and an actuator. The needle holder defines a needle-receiving passage that holds a needle. The actuator is configured such that movement from a first operating position toward a second operating position moves the needle in an advance direction. The actuator includes a button and a spring biasing the actuator toward the second operating position. The button is operatively connected with the spring so as to preclude the spring from moving the actuator toward the second operating position until after the button has been moved from a non-actuated position toward an actuated position. The suturing device disclosed in U.S. Pat. No. 10,610,215 B2 is particularly well suited repairing a tear in the dura mater. When, however, repairing denser tissue, modifications to such a device are desired.

U.S. Pat. No. 5,578,044 A discloses a suture application system including a cannular body comprised of housing halves that when connected form an opening and a suture slot. The suture slot appears to be curved. Moreover, when read in context with previously described embodiments, the suture slot appears to be aligned with a plane in which a curved needle groove is provided. Locating the suture slot in this plane may result in issues when deploying a curved needle using a flexible needle driver to drive the curved needle through the curved needle groove.

### SUMMARY

In view of the foregoing, a suturing device includes a cannula, a needle pusher and an actuator mechanism. The cannula includes a needle compartment defining a needle-receiving passage terminating at a distal opening of the cannula and a notch extending from the distal opening into the cannula. The needle pusher includes a wire provided in the cannula. The actuator mechanism operably connects with the needle pusher such that actuation of the actuator mechanism results in movement of the wire within the cannula toward the distal opening.

A method of assembling the suturing device includes inserting a needle having a suture attached thereto through the distal opening into the needle - receiving passage with a portion of the suture received in the notch and another portion of the suture extending away from the cannula. The suture can be pulled along the notch to move the needle in a retract direction toward a retracted position in which the needle is retained in the needle-receiving passage.

### BRIEF DESCRIPTION OF THE DRAWINGS

FIG. 1 is a perspective view of a suturing device.

FIG. 2 is a cross-sectional view of the suturing device of FIG. 1.

FIG. 3 is a cross-sectional view of a distal portion of the suturing device of FIG. 1 with a needle shown in a retracted position.

FIG. 4 is a side view of the distal portion of the suturing device of FIG. 1 with the needle shown in the retracted position.

FIG. 5 is a side view of the distal portion of the suturing device of FIG. 1 with the needle shown being deployed from the suturing device.

FIG. 6 is a cross - sectional view taken through line 6-6 in FIG. 4.

FIG. 7 is a side view of a distal portion of an alternative suturing device having an actuator mechanism similar to that shown in FIG. 2 with a needle shown in a retracted position.

### DETAILED DESCRIPTION

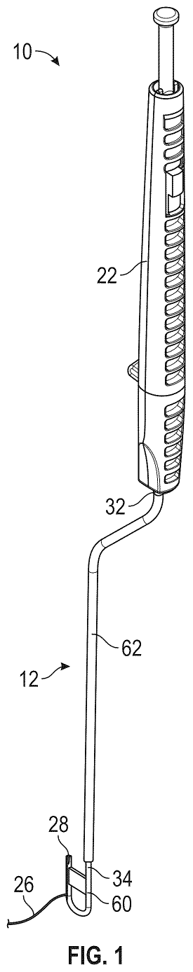

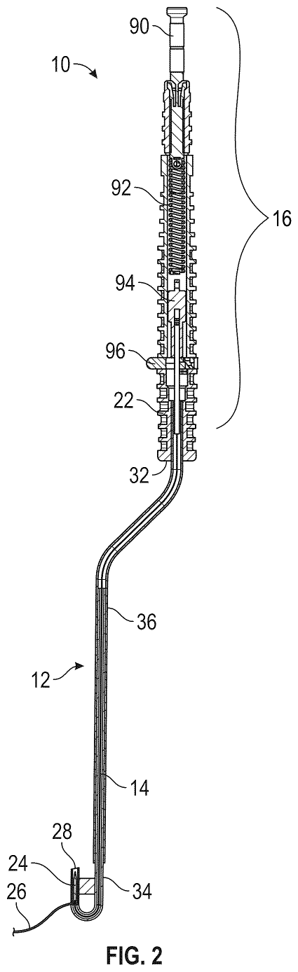

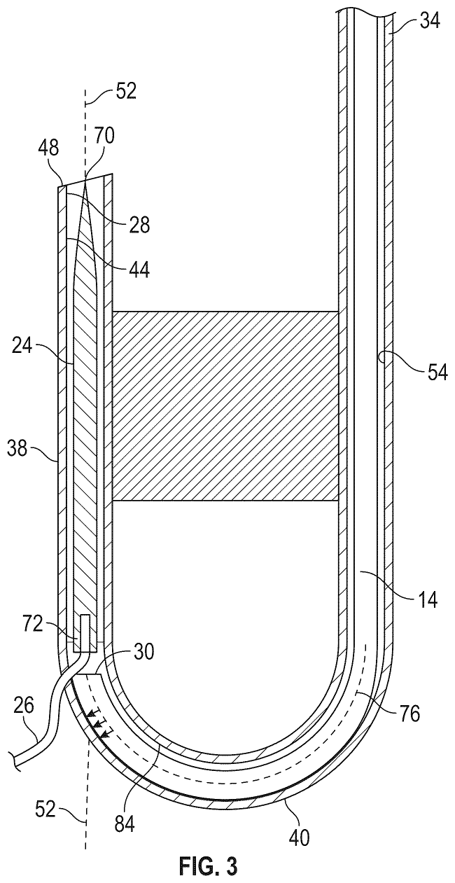

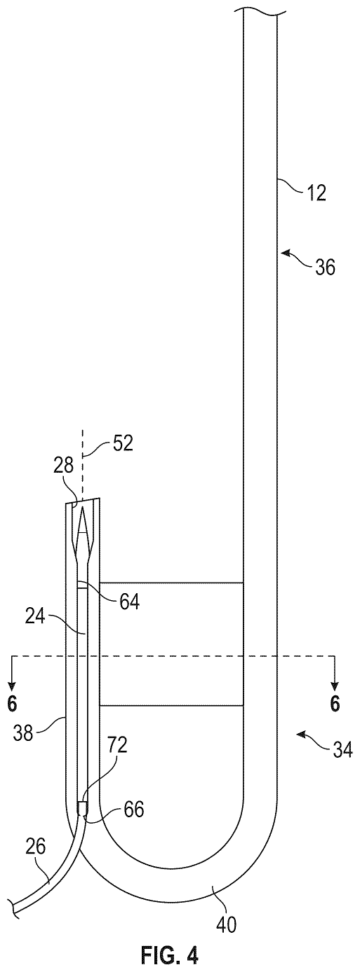

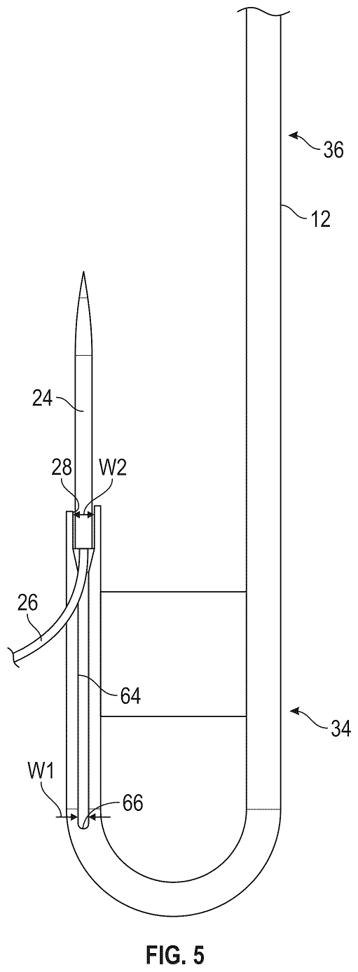

FIGS. 1 and 2 depict a suturing device 10 including a cannula 12, a needle pusher 14 and an actuator mechanism 16. The suturing device 10 can further include a handle 22 connected with the cannula 12. A needle 24 and a suture 26 connected with the needle 24 are loaded into the suturing device 10 allowing the suturing device 10 to be useful to repair tissue tears. The actuator mechanism 16 operably connects with the needle pusher 14. With reference also to FIGS. 3 and 4, movement of the needle pusher 14 within the cannula 12 in an advance direction moves the needle 24 toward a distal opening 28 of the cannula 12, which results in the needle 24 and the suture 26 being released from the suturing device 10. FIG. 5 depicts a distal end 30 of the needle pusher 14 having been moved closer toward the distal opening 28 as compared to FIG. 4 to push the needle 24 through the distal opening 28. The needle pusher 14 can also be moved within the cannula 12 in a retract direction to move the distal end 30 of the needle pusher 14 away from the distal opening 28 of the cannula 12 to allow another needle to be loaded into the suturing device 10.

With reference to FIGS. 1 and 2, the cannula 12 includes a proximal portion 32, a distal portion 34 and a main shaft 36. The proximal portion 32 is located nearer to and/or can be partially received in the handle 22. The main shaft 36 is positioned between the proximal portion 32 and the distal portion 34 and includes a relatively straight section that can facilitate insertion into a portal, such as a tubular retractor, but could also take other configurations for insertion into other passages. As more clearly seen in FIG. 3, the distal portion 34 includes a needle compartment 38 and a curved section 40. The needle compartment 38 defines a needle-receiving passage 44 terminating in the distal opening 28 of the cannula 12. As illustrated, the needle receiving passage 44 extends along a straight linear needle-receiving passage axis 52 to accommodate the needle 24, which is a straight needle in the embodiment depicted in FIG. 3. The needle-receiving passage axis 52 is a straight linear axis from the distal opening 28 to where the needle compartment 38 transitions to the curved section 40. The curved section 40 is adjacent to the needle compartment 38. The curved section 40 transitions into the needle compartment 38 at an end of the needle compartment 38 that is opposite the distal opening 28. A distal edge surface 48 of the needle compartment 38 defines the distal opening 28. As more clearly seen in FIG. 2, the distal edge surface 48 of the needle compartment 38 slopes upwardly toward the proximal portion 32 of the cannula 12.

The cannula 12 defines a needle pusher passage 54 in which the needle pusher 14 is received prior to actuation of the actuator mechanism 16. FIG. 3 depicts the needle pusher 14 prior to actuation of the actuator mechanism 16. As illustrated, the curved section 40 has an arc length of 180 degrees and the needle pusher passage 54 extends into the curved section 40. In alternative configurations, the curved section 40 can have other arc lengths, e.g., between 150 degrees and 210 degrees. The needle-receiving passage 44 connects with the needle pusher passage 54 to provide a continuous passage within the cannula 12 allowing the needle pusher 14 to move into the needle-receiving passage 44 as the needle pusher 14 drives the needle 24 in the advance direction toward the distal opening 28.

As illustrated, and with reference back to FIG. 1, the cannula 12 includes an inner cannula 60 received in an outer cannula 62 where the distal portion 34 of the cannula 12 is provided on the inner cannula 60. Nevertheless, it is contemplated that the cannula 12 can be made from a single tube or more than two tubes connected with one another. In the depicted embodiments, the cannula 12 is circular in a cross section taken normal to the longest dimension of the cannula 12, however, the cannula 12 could take alternative configurations in cross section, such as polygonal. The cannula 12 has a bayonet configuration in the illustrated embodiment; however, the cannula 12 could take alternative configurations, such as straight along a longitudinal axis.

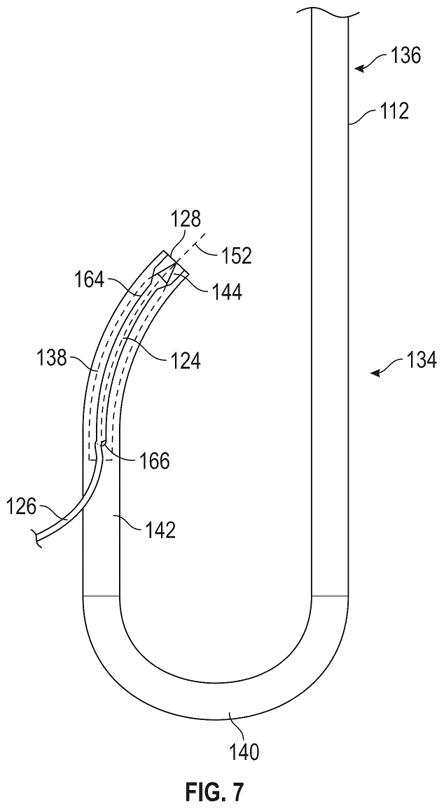

With reference to FIG. 4, the cannula 12 further includes a notch 64 extending from the distal opening 28 into the cannula 12. The notch 64 is configured to receive the suture 26 attached to the needle 24. In the illustrated embodiment, the notch 64 is provided in the inner cannula 60. In the example shown in FIG. 4, the notch 64 extends in a straight linear direction (parallel to the linear needle-receiving passage axis 52) into the cannula 12 from the distal opening 28 to a proximal end 66 of the notch 64. As illustrated, the notch 64 extends from the distal opening 28 into the cannula 12 a distance that is slightly longer than the length of the needle 24. The notch 64 can extend from the distal opening 28 into the cannula 12 a distance that is at least greater than a majority of the length of the needle 24 while not being as long as the needle 24. Such a construction, which is shown in FIG. 7 for an alternatively shaped needle, can facilitate both retention of the needle 24 and its release during deployment, which will be described in more detail below. In FIG. 4, the notch 64 extends into the curved section 40 of the cannula 12 while still extending in the straight linear direction from the distal opening 28 to the proximal end 66, which is located within the curved section 40. If desired, the notch 64 could extend from the distal opening 28 and terminate at the proximal end 66 adjacent to the curved section 40 while not located within the curved section 40. The notch 64 widens adjacent to the distal opening 28. The notch 64 has a first width W1 adjacent to the proximal end 66 and a second width W2 adjacent to the distal opening 28. Each width W1, W2 is measured perpendicular to a needle-receiving passage axis 52 and the first width is less than the second width. The suture 26 has an outer diameter (OD) less than both the first width W1 and the second width W2.

The needle pusher 14 can include a wire, which can be made from nitinol. The wire of the needle pusher 14 has an outer diameter (OD) greater than the first width W1 and less than the second width W2. With reference to FIG. 3, the needle 24 includes a first end 70, which is pointed, and a second end 72 that is opposite the first end 70. The suture 26 connects with the second end 72 of the needle 24. In the illustrated embodiment, at least a portion of the suture 26 extends through the notch 64 when the needle 24 is received in the needle compartment 38 prior to the actuator mechanism 16 being actuated. Prior to actuation of the actuator mechanism 16, the distal end 30 of the needle pusher 14 can be offset from the second end 72 of the needle 24. The needle pusher passage 54 extends into the curved section 40 of the cannula 12, and prior to actuation of the actuator mechanism 16 a portion of the needle pusher 14 extends into the curved section 40 of the distal portion 34 of the cannula 12.

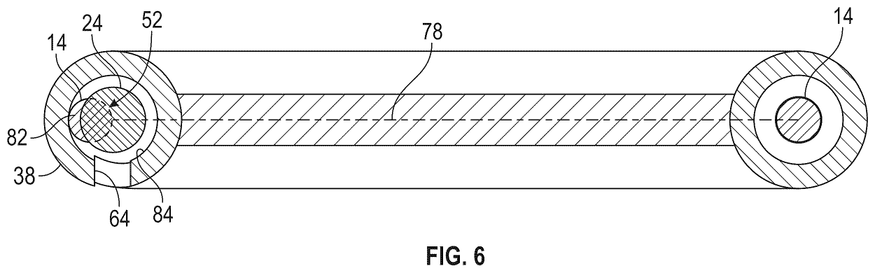

Upon actuation of the actuator mechanism 16, which will be described in more detail below, the needle pusher 14 moves through the curved section 40 to push the needle 24 in the advance direction through the distal opening 28. Actuation of the actuator mechanism 16 results in movement of the wire of the needle pusher 14 within the cannula 12 along a curve 76 (FIG. 3) within the curved section 40 and the notch 64 is offset from a plane 78 (FIG. 6) defined by the curve 76. In FIG. 6, the plane 78 is depicted as a line, but the plane 78 is normal to the cross-section depicted in FIG. 6. Having the notch 64 offset from a plane 78 is useful to decrease the likelihood of the needle pusher 14 getting caught within the notch 64 as the wire of the needle pusher 14 moves within the needle-receiving passage 44. With reference to FIGS. 3 and 6, the wire of the needle pusher 14 imparts an outwardly directed force (see arrows depicted in FIG. 3) on a portion 82 (FIG. 6) an inner wall surface 84 (FIG. 6) of the cannula 12 as the needle pusher 14 moves within the needle compartment 38 and the curved section 40 in the advance direction. The notch 64 is located angularly offset about the needle-receiving passage axis 52 from the portion 82 of the inner wall surface 84 against which the outwardly directed force is imparted in a cross section taken normal to the needle-receiving passage axis 52. The inner wall surface 84 having the C-shape in the cross section shown in FIG. 6 discourages the needle pusher 14 from traveling towards the notch 64 as the needle pusher 14 moves within the needle-receiving passage 44 thus inhibiting the needle pusher 14 from getting caught within the notch 64.

With reference to FIG. 2, the actuator mechanism 16 is operably connected with the needle pusher 14. The actuator mechanism 16 includes a plunger 90, a spring 92, a shuttle 94, and a button 96. The plunger 90 is movable between an extended position and a depressed position. When the button 96 is not depressed, which is the position shown in FIG. 2, movement of the plunger 90 from the extended position toward the depressed position compresses the spring 92 against the shuttle 94. The spring 92 exerts a biasing force on the shuttle 94 in the advance direction when the plunger 90 is in the depressed position. The button 96 is operably engaged with the shuttle 94. The spring 92 biases the shuttle 94 in the advance direction after the button 96 is depressed, which moves the needle pusher 14 and pushes the needle 24 in the advance direction. Further explanation of the actuator mechanism is provided in U.S. application Ser. No. 18/150,868; however, other actuator mechanisms capable of moving the needle pusher 14 and pushes the needle 24 in the advance direction. For example, U.S. Pat. No. 10,9183,79 B2 describes another type of actuator mechanism that could be employed to move the needle pusher 14 and push the needle 24 in the advance direction.

Providing the suturing device 10 described above allows for easy assembly and reloading of the suturing device 10. To load the suturing device 10 with the needle 24 and the suture 26, the needle pusher 14 is moved to the state shown in FIG. 3 and the needle 24 is inserted through the distal opening 28 into the needle-receiving passage 44 with a portion of the suture 26 received in the notch 64 and another portion of the suture 26 extending away from the cannula 12. The suture 26 is then pulled (downward per the orientation shown in FIG. 4) along the notch 64 to move the needle 24 in the retract direction toward a retracted position (shown in FIG. 3) in which the needle 24 is retained in the needle-receiving passage 44. In the example shown in FIGS. 3 and 4, the notch 64 extends in a straight linear direction into the cannula 12 from the distal opening 28 to the proximal end 66 of the notch 64. With this construction, pulling the suture 26 along the notch 64 results in a portion of the suture 26 in contact with the cannula 12 within the notch 64 moving in the retract direction and along the straight linear direction. It can be desirable to pull the suture 26 along the notch 64 until the first end 70 of the needle 24 is offset from the distal opening 28 when in the retracted position as shown in FIG. 3. The suture 26 can also be pulled along the notch 64 until the second end 72 of the needle 24 is offset from the proximal end 66 of the notch 64 in the advance direction, which is visible in FIG. 4. The suture 26 can also be pulled along the notch 64 until the second end 72 of the needle 24 is positioned in the curved section 40, which is visible in FIG. 3.

Having the notch 64 extend from the distal opening 28 into the cannula 12 at least a majority of the length of the needle 24 can be particularly useful when the suture 26 is a braided suture. A braided suture, acting like a rope, is primarily designed to handle tensile loads or forces along its length. When tension is applied, the individual strands of the braid distribute the load, making it suitable for applications such as stitching tissue together or carrying bridge loads. However, the structure of the braided suture is not optimized for compressive forces. The following factors make compressing a braided suture difficult. Braided sutures are composed of intertwined fibers arranged in a specific pattern to enhance tensile strength. When a compressive load is applied the braided suture, the individual fibers tend to buckle and cannot efficiently distribute the compressive load. This makes braided sutures less effective in transmitting a compressive force as compared to a monofilament suture, which acts more like a rigid body. Also, the interaction between the strands in a braided suture is designed to provide flexibility and strength under tension. However, this interstrand interaction can hinder the ability of the suture to withstand compression. The friction between strands can cause them to slide against each other rather than transmitting the compressive force along the length of the braided suture, which has been referred to as “unravelling.” Unlike rigid bodies that transmit loads directly, the intertwined fibers in a braided suture distribute loads across multiple elements making it effectively a multi-body system.

If the notch 64 is not provided in the cannula, for a needle 24 having a length of about 12 mm, the suture 26 would extend through the distal opening 28 and fold into the cannula 12 before attaching to the second end 72 of the needle 24. With this construction with the notch 64, when the needle 24 is being deployed through tissue, the suture 26 is constrained by the underside of the tissue, which compresses the suture 26 and promotes bunching. By providing the notch 64 extending from the distal opening 28 into the cannula 12 at least a majority of the length of the needle 24, the suture 26 can be offset from the distal opening 28 during deployment and thus the underside of the tissue as the needle 24 is being driven through the tissue so that the only drag of the suture 26 is the drag as the suture 26 pulls through the tissue after the needle 24 is fully deployed.

FIG. 7 shows a side view of a lower portion of a main shaft 136 and a distal portion 134 of an alternative suturing device having an actuator mechanism similar to that shown in FIG. 2. Accordingly, the actuator mechanism will not be described again in detail. FIG. 7 depicts an alternative needle 124, which in this example includes a curvature, shown in a retracted position. Similar to the embodiment depicted in FIGS. 1-6, the distal portion 134 includes a needle compartment 138 and a curved section 140. The distal portion 134 also includes a straight section 142 between the needle compartment 138 and the curved section 140.

The needle compartment 138 defines a needle-receiving passage 144 terminating at a distal opening 128 of a cannula 112. The cannula 112 is similar to the cannula 12 described above, and also receives a needle pusher (not illustrated, but similar to the needle pusher 14 described above). The needle-receiving passage 144 extends along a curved needle-receiving passage axis 152 to accommodate the needle 124, which has a curvature matching the curved needle-receiving passage axis 152 in the embodiment depicted in FIG. 7. The curved needle-receiving passage axis 152 extends from the distal opening 128 to where the needle compartment 138 transitions to the straight section 142. The curved section 140 transitions into the straight section 142 at an end of the straight section 142 that is opposite the distal opening 128.

The cannula 112 further includes a notch 164 extending from the distal opening 128 into the cannula 112. The notch 164 is configured to receive a suture 126 attached to the needle 124. In the example shown in FIG. 7, the notch 164 extends along following the curvature of the curved needle-receiving passage axis 152 into the cannula 112 from the distal opening 128 to a proximal end 166 of the notch 164. The notch 164 extends from the distal opening 128 into the cannula 112 a distance at least 90% of the length of the needle 124. As illustrated, the notch 164 extends from the distal opening 128 into the cannula 112 a distance that is slightly less than the length of the needle 124. Similar to the example described above, the notch 164 can extend from the distal opening 128 into the cannula 112 a distance that is at least greater than a majority of the length of the needle 124 while not being as long as the needle 124. Such a construction, which is shown in FIG. 7, can facilitate both retention of the needle 124 and its release during deployment. In FIG. 7, the notch 164 extends into the straight section 142 of the cannula 112. If desired, the notch 164 could extend from the distal opening 128 and terminate at the proximal end 166 without extending into the straight section 142. The notch 164 widens adjacent to the distal opening 128 similar to the notch 64 described above. The suture 26 has an outer diameter (OD) less than the width of the notch 164.

The notch 164 in the example shown in FIG. 7 is offset from a plane (similar to the plane 78 shown in FIG. 6) defined by the curve of the curved section 140. The needle 124 can be deployed in a similar manner to the needle 24 described above. The example depicted in FIG. 7 is similar in many respects to the example described with reference to FIGS. 1-6; however, the needle-receiving passage 144 extends along the curved needle-receiving passage axis 152 towards the main shaft 136 when moving along the advance direction, which is the direction in which the needle 124 travels when being deployed. Such a configuration can allow for a better condition when deploying the needle 124 by directing the needle 124 towards the main shaft 136 instead of outwardly away from or straight up with respect to the main shaft 136 where it may impinge on other tissue.

It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A suturing device comprising:
a cannula including a main shaft and a distal portion having a needle compartment, a curved section and a notch, the needle compartment defining a needle-receiving passage terminating at a distal opening of the cannula and configured to receive a needle and the notch extending into the cannula from the distal opening and configured to receive a suture attached to the needle;
a needle pusher including a wire provided in the cannula; and
an actuator mechanism operably connected with the needle pusher, wherein actuation of the actuator mechanism results in movement of the wire in an advance direction within the cannula along a curve within the curved section and the notch being offset from a plane defined by the curve, and the needle compartment being configured such that movement of the wire in the advance direction pushes the needle through the distal opening parallel to or towards the main shaft.

2. The suturing device of claim 1, wherein the wire imparts an outwardly directed force on a portion an inner wall surface of the cannula as the wire moves within the curved section toward in the advance direction and the notch is located angularly offset about a needle-receiving passage axis defined by the needle-receiving passage from the portion of the inner wall surface against which the outwardly directed force is imparted in a cross section taken normal to the needle-receiving passage axis.

3. The suturing device of claim 2, wherein the notch extends along a straight linear direction from the distal opening and terminates at a proximal end adjacent to or within the curved section.

4. The suturing device of claim 3, wherein the notch terminates within the curved section.

5. The suturing device of claim 3, wherein the needle-receiving passage axis is a straight linear axis from the distal opening to where the needle compartment transitions to the curved section.

6. The suturing device of claim 1, wherein the needle-receiving passage axis is a curved needle-receiving passage axis.

7. The suturing device of claim 6, wherein the notch extends along and follows a curvature of the curved needle-receiving passage axis.

8. The suturing device of claim 7, wherein the needle-receiving passage extends along the curved needle-receiving passage axis towards the main shaft when moving along the advance direction.

9. The suturing device of claim 7, wherein the distal portion also includes a straight section between the needle compartment and the curved section.

10. The suturing device of claim 1, wherein the notch has a first width adjacent to a proximal end of the notch spaced from the distal opening and a second width adjacent to the distal opening, wherein each width is measured perpendicular to a needle-receiving passage axis defined by the needle-receiving passage and the first width is less than the second width.

11. The suturing device of claim 10, wherein the wire has an outer diameter (OD) greater than the first width and less than the second width.

12. A method of assembly comprising:
providing a suturing device including
a cannula including a needle compartment defining a needle - receiving passage terminating at a distal opening of the cannula, a curved section, and a notch extending from the distal opening into the cannula;
a needle pusher including a wire provided in the cannula; and
an actuator mechanism operably connected with the needle pusher, wherein actuation of the actuator mechanism results in movement of the wire in an advance direction within the cannula along a curve within the curved section and the notch being offset from a plane defined by the curve,
inserting a needle having a suture attached thereto through the distal opening into the needle-receiving passage with a portion of the suture received in the notch and another portion of the suture extending away from the cannula; and
pulling the suture along the notch to move the needle in a retract direction toward a retracted position in which the needle is retained in the needle-receiving passage.

13. The method of assembly of claim 12, wherein the notch extends in a straight linear direction into the cannula from the distal opening to a proximal end of the notch, and pulling the suture along the notch results in a portion of the suture in contact with the cannula within the notch moving in the retract direction and along the straight linear direction.

14. The method of assembly of claim 12, wherein the notch extends from the distal opening into the cannula a distance at least 90% of the length of the needle, and pulling the suture along the notch includes pulling the suture until a pointed end of the needle is offset from the distal opening in the retracted position.

15. The method of assembly of claim 14, wherein pulling the suture along the notch further includes pulling the suture until a second end of the needle, which is opposite the pointed end, is offset from a proximal end of the notch in the advance direction.

16. The method of assembly of claim 12, wherein the cannula includes a curved section adjacent to the needle compartment and the notch extends in a straight linear direction from the distal opening into the curved section, wherein pulling the suture along the notch further includes pulling the suture until a second end of the needle, which is opposite a pointed end, is positioned in the curved section.

17. The method of assembly of claim 12, wherein the suture is a braided suture.
